(19) United States
(12) Patent Application Publication
(10) Pub. No.: US 2024/0312094 A1
(43) Pub. Date: Sep. 19, 2024

(54) Transcriptive Biomechanical System And Method

(71) Applicant: Odd Thinking LLC, Seattle, WA (US)

(72) Inventors: Mark Carr, Mukilteo, WA (US); Heather Kain, Seattle, WA (US); Mustafa Ramadan, Kent, WA (US); Humza Mangrio, Auburn, WA (US)

(21) Appl. No.: 18/607,290

(22) Filed: Mar. 15, 2024

Related U.S. Application Data
(60) Provisional application No. 63/452,626, filed on Mar. 16, 2023.

(51) Int. Cl. G06T 13/20; A61B 5/00; G06T 13/40; G06T 17/00; G10L 17/02; G10L 17/26; G10L 21/0208; G10L 25/60; G10L 25/66; G10L 25/93

(52) U.S. Cl. CPC G06T 13/205; A61B 5/4088; A61B 5/4803; G06T 13/40; G06T 17/00; G10L 17/02; G10L 17/26; G10L 21/0208; G10L 25/60; G10L 25/66; G10L 25/93; G06T 2210/41

(57) ABSTRACT

Embodiments described herein include aspects related generating a biomechanical data output. In one embodiment, the lexical data from input data is generated using codex mapping, the lexical data including multiple sound units. A stack of 3-Dimensional objects is generated using a biomechanical model for each sound unit in the lexical data, wherein the 3-Dimensional objects include components from the biomechanical model. An animated biomechanical data output is generated by rigging the stack of 3-Dimensional objects such that the animated biomechanical model of the interaction of the elements produce the lexical data.

### BACKGROUND

With speech data becoming more abundant it is increasingly important to understand “language” as a sequence of acoustic events, rather than a byproduct of Linguistics subfields alone, because it better suits modern machine learning models that use spectral and spectrographic data to learn acoustic events. Furthermore, transcribing acoustic data as a biomechanical system of anatomical landmarks that follow a path, such as a digitized vocal apparatus moving across coordinate space when activated by the audio input of a speaker, can augment acoustic or linguistic intelligence, especially when used in tandem with syntax trees, semantic question-and-answering, or similar natural language techniques.

With this patent we produce a transcriptive process to translate acoustic, linguistic, and biomechanical intelligence by way of a codex, and from this codex a stream of data can optionally control a digital biological apparatus that moves across coordinate space. The shape of the biological apparatus also comes from a codex translation, as does any translation from one model output to another.

This technology is useful for systems creating synthetic data, for medical professionals who need to understand disorders based on acoustics and biomechanics, and it serves as a tool for further generative data modeling. The biomechanical data is essentially reverse-engineered kinematic motion tracking data, so the generated output is easily “rigged” to digital objects that can move. For instance, the codex might use International Phonetic Alphabet chart lookups to drive a diagram of the vocal apparatus that changes with each phoneme. Generating spatial data from acoustics becomes especially powerful in machine learning when sequenced with other multimodal inputs, like language or vision.

Acoustic data is produced by delicate and complex processes that come from biological systems. Why are we not creating a codex to encode and decode these moving biological systems as spatial data for greater acoustic understanding while we sequence the language, much like human perception would allow? A system and method is needed to improve the technology by generating biomechanical data based on a codex and letting each type of model therein augment the other.

### SUMMARY

Embodiments of the present disclosure relate to, among other things, a system and method to generate biomechanical data and augment acoustic understanding of through transcription and coding of associated data. In particular, embodiments described herein obtain input data. If the data is not presently usable acoustic data, the transcriptive system processes start by cleaning, formatting and engineering features that process those inputs. The transcriptive biomechanical system further augments acoustic understanding and formats syntactic, lexical, phonetic, etc. data as a biomechanical system by using the codex. Finally the data can animate a visual output or produce a raw stream of data. The codex might take acoustic data and move a 3-Dimensional stack of objects, or it might simply answer a pragmatic question. The 3D stack of objects are “rigged” to produce an animated biomechanical output across sets of coordinates.

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Additional objects, advantages, and novel features of the technology will be set forth in part in the description which follows, and in part will become apparent to those skilled in the art upon examination of the disclosure or learned through practice of the technology.

### BRIEF DESCRIPTION OF THE DRAWINGS

The present technology is described in detail below with reference to the attached drawing figures, wherein:

FIG. 1 is a diagram of an environment that can be used to perform the transcriptive biomechanical method, according to embodiments of the present disclosure

FIG. 2 provides an example transcriptive biomechanical system as it pertains to the codex, in accordance with embodiments described herein.

FIG. 3 is a flow diagram illustrating an exemplary method for generating biomechanical data and other exemplary codex data in accordance with one embodiment of the present disclosure.

FIG. 4 is an exemplary operating environment for implementing embodiments of the present technology.

### DETAILED DESCRIPTION

With advancement of technology, a huge amount of data is being processed and transmitted throughout the world. A good chunk of that data being processed and transmitted is acoustic data, which is the central “data input” we are describing herein. Commercial digital platforms and social networks allow users to broadcast acoustic and video data, and this data can be accessed by millions of viewers worldwide within seconds. Transcribing the acoustic data from this media allows users from all over the world to create a digital copy of the “performance.” Understanding acoustics will help the unfamiliarity introduced by speaker accents, and the technology can help with translation by augmenting other language systems. Using a codex to derive this sort of data also allows users unable to access acoustic data because of impairment to have augment their experience. Transcription might also include the process of converting acoustic data into natural language features.

Conventional systems have various deficiencies. For example, existing technologies that transcribe speech data are not accurate. Most conventional systems are designed to recognize acoustics as language from models with biased training datasets, but this system alleviates the difficulties of language barriers by understanding acoustics as a biological system in motion. For example, most conventional speech-to-text systems recognize English-speaking, North American accents with greater accuracy than other accents. Complicating matters, conventional speech-to-text systems are unable to accurately transcribe acoustic data if the speaker changes speed, or if there are multiple speakers with overlapping dialogue, or if other far field noises are causing problems. The quality of acoustic data, or the noise therein due to the environment (e.g. walking around while speaking, ambience, wind, etc.), can drop the accuracy of transcription significantly. Because acoustic data is often produced by delicate and complex processes from biological systems, a system and method is needed in order to understand the voice as a biological system in motion. The biomechanical data generated can from the codex translation can also help validate conventional acoustic or language systems.

Various aspects of the present disclosure improve these existing technologies by providing one or more technical solutions to one or more of the technical problems described herein. In operation the system first processes acoustic input data from a user. The data processing can include time stamps, linguistic feature engineering, and text and audio processing. The codex that drives this system uses a variety of machine learning models to understand acoustic, biomechanical, and linguistic data being transcribed and translated. The system uses the codex to map the acoustic and language data to a digital biological “body.” A user might be mapping acoustic, phonetic, and text data to lexical data using codex. Whatever combinations of translations, the codex will ultimately deliver the biomechincal rigging. For example, if the biomechanical model is of a human, then the system contains various components such as lungs, trachea, voice box, muscles, mouth, tongue, lips, and the like. The system can illustrate how motion or sound is created from the movement of these components. The system illustrates how air was flowing through the lungs, through the trachea, voice box that causes the voice box to vibrate and produce the acoustic data that was captured, transcribed, and translated. The biomechanical model can further illustrate how the muscles were controlling the soft palate, tongue and lips to produce the sound. The system arranges a 3D stack of objects replicating the body system and produces an animated biomechanical output.

Various aspects of the present disclosure improve existing language models and computer applications. For example, the system can identify irregularity by determining if the biomechanical motion is even humanly feasible. Or conversely it can “fill in the blank” when text interprets audio as “inaudible,” by providing the biological function that was non-language. After generating the 3D biomechanical motion, if the system identifies that there is long pauses from memory lapses or slurring, the system can identify that as an early symptom of dementia and the user can see how biological functions are changing. Other conditions can be recognized based on different irregularities. It should be understood that any other part of the system can be analyzed to determine an underlying medical condition.

Accordingly, some embodiments of the present disclosure are directed to employing techniques for generating biomechanical data based on receiving input data. In one example, the input data can be received from different sources rather than just acoustic ones. Text will also work, and eventually video will as well. The transcriptive biomechanical system receives data, and if the received input data is not proper acoustic data format, the transcriptive biomechanical system processes the input data into a proper data format. The transcriptive biomechanical system uses translated data to produce 3-Dimensional (3D) stack of objects that are rigged together and produce motion across coordinates.

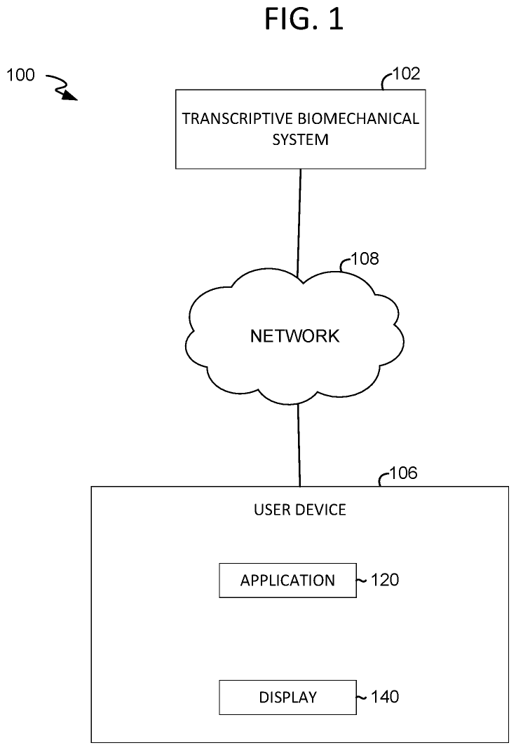

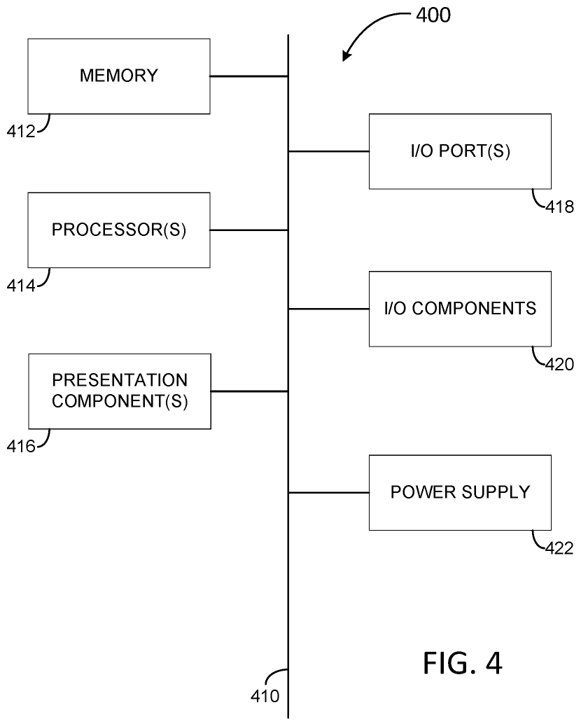

Turning to FIG. 1, FIG. 1 is a diagram of an environment 100 that can be used to perform the transcriptive biomechanical method, according to embodiments of the present disclosure. It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether for the sake of clarity. Further, many of the elements described herein are functional entities that may be implemented as discrete or distributed components or in conjunction with other components, and in any suitable combination and location. Various functions described herein as being performed by one or more entities may be carried out by hardware, firmware, and/or software. For instance, some functions may be carried out by a processor executing instructions stored in memory as further described with reference to FIG. 4.

The system 100 is an example of a suitable architecture for implementing certain aspects of the present disclosure. In one embodiment, the system 100 includes, among other components not shown, a transcriptive biomechanical system 102, and a user device 106. Each of the transcriptive biomechanical system 102 and user device 106 shown in FIG. 1 can comprise one or more computer devices, such as the computing device 400 of FIG. 4 discussed below. The transcriptive biomechanical system 102 may be embodied at least partially by the instructions corresponding to application 120. Therefore, the transcriptive biomechanical system 102 can operate on a server or on a user device, such as user device 106, or partially on multiple devices. As shown in FIG. 1, the transcriptive biomechanical system 102 and the user device 106 can communicate via a network 108, which may include, without limitation, one or more local area networks (LANs) and/or wide area networks (WANs). Such networking environments are commonplace in offices, enterprise-wide computer networks, intranets, and the Internet. It should be understood that any number of user devices and transcriptive biomechanical systems may be employed within the system 100 within the scope of the present disclosure. Each may comprise a single device or multiple devices cooperating in a distributed environment. For instance, the transcriptive biomechanical system 102 could be provided by multiple devices collectively providing the functionality of the transcriptive biomechanical system 102 as described herein. Additionally, other components not shown may also be included within the network environment.

It should be understood that any number of user devices 106, transcriptive biomechanical systems 102, and other components can be employed within the operating environment 100 within the scope of the present disclosure. Each can comprise a single device or multiple devices cooperating in a distributed environment.

User device 106 can be any type of computing device capable of being operated by a user. For example, in some implementations, user device 106 is the type of computing device described in relation to FIG. 4. By way of example and not limitation, a user device 106 may be embodied as a personal computer (PC), a laptop computer, a mobile device, a smartphone, a tablet computer, a smart watch, a wearable computer, a personal digital assistant (PDA), an MP3 player, a global positioning system (GPS) or device, a video player, a handheld communications device, a gaming device or system, an entertainment system, a vehicle computer system, an embedded system controller, a remote control, an appliance, a consumer electronic device, a workstation, any combination of these delineated devices, or any other suitable device.

The user device 106 can include one or more processors, and one or more computer-readable media. The computer-readable media may include computer-readable instructions executable by the one or more processors. The instructions may be embodied by one or more applications, such as application 120 shown in FIG. 1. Application 120 is referred to as a single application for simplicity, but its functionality can be embodied by one or more applications in practice. As indicated above, the other user devices can include one or more applications similar to application 120.

The application(s) may generally be any application capable of facilitating generation of biomechanical data (e.g., via the exchange of information between the user devices and the transcriptive biomechanical system 102). In some implementations, the application(s) comprises a web application, which can run in a web browser, and could be hosted at least partially on the server-side of environment 100. In addition, or instead, the application(s) can comprise a dedicated application, such as an application having image processing functionality. In some cases, the application is integrated into the operating system (e.g., as a service). It is therefore contemplated herein that “application” be interpreted broadly.

In accordance with embodiments herein, the application 120 can either initiate the transcriptive biomechanical system 102 to facilitate transcriptive biomechanical method via a set of operations initiated to display the generated biomechanical data output on a display 140 of to the user device 106.

In embodiments, the transcriptive biomechanical system 102, receives input data and verifies whether the received input data is in an acoustic data format. If the received input data is not in an acoustic data format, the transcriptive biomechanical system 102 processes the received input data to convert the data into acoustic data. Processing the received input data can involve, among other things, filtering the data to remove background noise or any unwanted noise, segmenting the received input data into window sizes and frames, identifying the number of speakers and the type of acoustic generators (human, dog, cat, whale etc.) or other factors. The received input data can be classified into chatter or speech and silence or any other identifiers.

The transcriptive biomechanical system 102 analyzes the acoustic data and produces new data using codex mapping. The transcriptive biomechanical system 102 maps the data into a biomechanical model to generate a stack 3-Dimensional (3D) objects related to the biomechanical model. The stack of 3D biomechanical objects include different biomechanical parts that work together to produce the sound. The stack of 3D objects is generated in a multi-dimensional coordinate system such as a x, y, z coordinate system. The transcriptive biomechanical system 102 performs rigging on the stack of 3D objects to produce an animated biomechanical data output of the biomechanical data.

For cloud-based implementations, the instructions on transcriptive biomechanical system 102 may implement one or more aspects of the transcriptive biomechanical system 102, and application 120 may be utilized by a user and/or system to interface with the functionality implemented on server(s). In some cases, application 120 comprises a web browser. In other cases, transcriptive biomechanical system 102 may not be required. For example, the functionality described in relation to the transcriptive biomechanical system 102 can be implemented completely on a user device, such as user device 106.

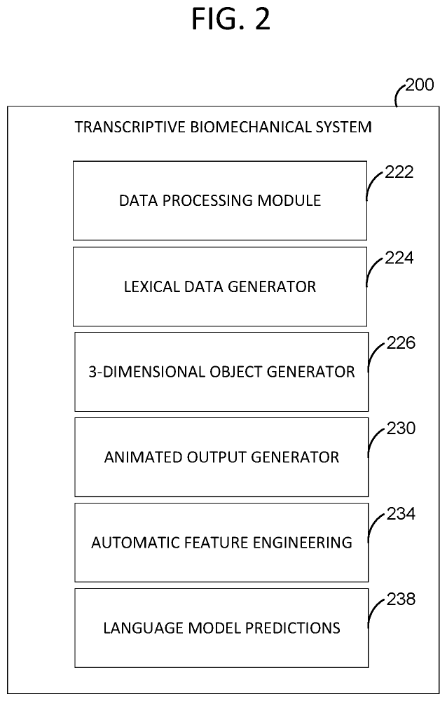

These components may be in addition to other components that provide further additional functions beyond the features described herein. The transcriptive biomechanical system 102 can be implemented using one or more devices, one or more platforms with corresponding application programming interfaces, cloud infrastructure, and the like. While the transcriptive biomechanical system 102 is shown separate from the user device 106 in the configuration of FIG. 1, it should be understood that in other configurations, some or all of the functions of the transcriptive biomechanical system 102 can be provided on the user device 106. Turning to FIG. 2, FIG. 2 provides an example transcriptive biomechanical system 200. As shown, an exemplary transcriptive biomechanical system 200 includes a data processing module 222, a lexical data generator 224, a 3-Dimensional (3D) object generator 226, and an animated biomechanical data output generator 230. As can be appreciated, any number of components may be used to perform the various functionalities described herein.

In accordance with the transcriptive biomechanical system 200, the data processing module 222 processes the received input data into an acoustic format. For example, speech-to-text functionality can be used to encode the input acoustic data into different aspects such as phonetic data, language data, and chunks of text. The data processing can include time stamps for data including acoustic, language data.

In some embodiments, the data processing module 222 receives input acoustic data. The data is then processed so that it can become data features and graphical representations understood by machines such as acoustic data, phonetic data, a first text data or the like. If the received input data is not in an acoustic data format, the transcriptive biomechanical system performs processing on the received input data to convert the data into acoustic data or biomechanical data. In some embodiments, the transcriptive biomechanical system can perform acoustic processing on the received input data in order to convert the received input data into an acceptable format that can be used in the transcriptive biomechanical system. In one example, processing the received input data into an acceptable format might include filtering the data to remove background noise or unwanted noise. In another example, processing the received input data can include segmenting the received input data into certain window sizes and even subdividing further. In another example, processing the received input data can further entail identifying the number of speakers or other biological systems generating acoustic or any systems generating acoustic. For example, the system can process the received input data to identify that multiple biosystems are making noise (such as two kids speaking and a bird chirping). In another example, processing can further identify subsets of speakers (e.g. infant, child, teen, adult, senior, male, female, bird, whale etc). It should be understood that further processing can be performed to prepare data for machine understanding. In some embodiments, the input data can be processed for better language understanding and the acoustic data can be modeled into a data to vector model that saves prominent information into memory for processing across all components of the codex.

In some embodiments the language data being generated in 224 converts the data from an acoustic format to a lexical data format using codex mapping. The lexical data generator 224 analyzes the input acoustic data or parsed acoustic data from the input data and produces motion of a vocal apparatus from a lexical understanding of phonemes, articulations, morphemes, and the like, as they relate to acoustic inputs, speech-to-text outputs, and biomechanical motion of the vocal apparatus. A “Codex” produces lookup codes that correspond to parts of the biological system (related to the vocal apparatus) and their individual motion tracing graphs and coordinates across vector space. Additional “parchments” (such as the rule book) provide holistic rulebooks, determining how each part interplays with the others, such as introducing rules for the holistic effect of displacement across the vocal apparatus. Lexical data may include data in the form of lexical semantics. Lexical semantics may be derived from the decomposition of acoustic into further sub-units of language or acoustic units for use as new data features. In some embodiments, the acoustic data is mapped to predefined lexical data and it creates motion. Sometimes acoustic data provides language understanding without being understood as a “language part” or something having a “global effect” on the animated representation of motion. Not all utterances are recognizable as words or parts of words. The codex mapping will also depend on the “biological parts” of the vocal apparatus. For example, if the acoustic being generated is by a human, and not a dog or cat, then the human speech found inside the codex will map the acoustic data to a coordinate system for a human vocal apparatus. If acoustic is being generated is by a bird then the codex will also map the lexical understanding to birds.

The 3D object generator 226 generates a stack of biomechanical objects. The generator 230 rigs the stack of biomechanical 3D objects to generate an animated biomechanical data output. In some embodiments, the 3D object generator 226 sends data to a biomechanical model and animates a “virtual stack” of 3-Dimensional (3D) objects, representing discrete parts of the biological apparatus during motion. These objects perform incremental motions from the acoustic inputs. For example, a stack of 3D biomechanical objects can include simulation of sound wave propagation in the 3D vocal track airway, movement of 3D vocal parts-all vibrating differently and variably based on airflow from the lungs, the movement of 3D lungs that generate the airflow, the movement of sound inside of a 3D nasal chamber, the movement of the 3D tongue, the movement of the 3D oropharyngeal to generate the sound in the lexical data, and the like. The stack of 3D biomechanical objects include different biomechanical parts that work together to produce the sound. The stack of 3D objects is generated in a multi-dimensional coordinate system such as a x, y, z coordinate system. It should be understood that stacks can be generated in any other dimension and any other coordinate system. In some embodiments, the 3D object generator 226 arrange the stack of 3D objects to vector space to produce an animated biomechanical output. In some embodiments, generating the stack of the 3-Dimensional objects includes obtaining and analyzing a stack of 3-Dimensional biological or biomechanical objects and the components producing motion or sound from the lexical understanding. Generating the stack of the 3-Dimensional objects further includes determining coordinates in a 3-Dimensional space for each 3-Dimensional biomechanical object by mapping each of the sound units to the lexical data and then to a stack of 3-Dimensional objects in a coordinate system, or just from acoustic data inputs alone.

In some embodiments, the animated biomechanical data output is used to validate transcripts generated from conventional systems, such as speech-to-text from video, similar media sources, or the like. By taking acoustic data from the transcriptive biomechanical system and a conventional system, the transcriptive biomechanical system can identify whether the transcripts from the conventional system is consistent with the transcriptive biomechanical system.

In one embodiment, the transcriptive biomechanical system output is used to analyze and understand how and why the biomechanical representations might be different.

In one embodiment, the transcriptive biomechanical system is used to analyze speech patterns and determine if the user has any medical condition.

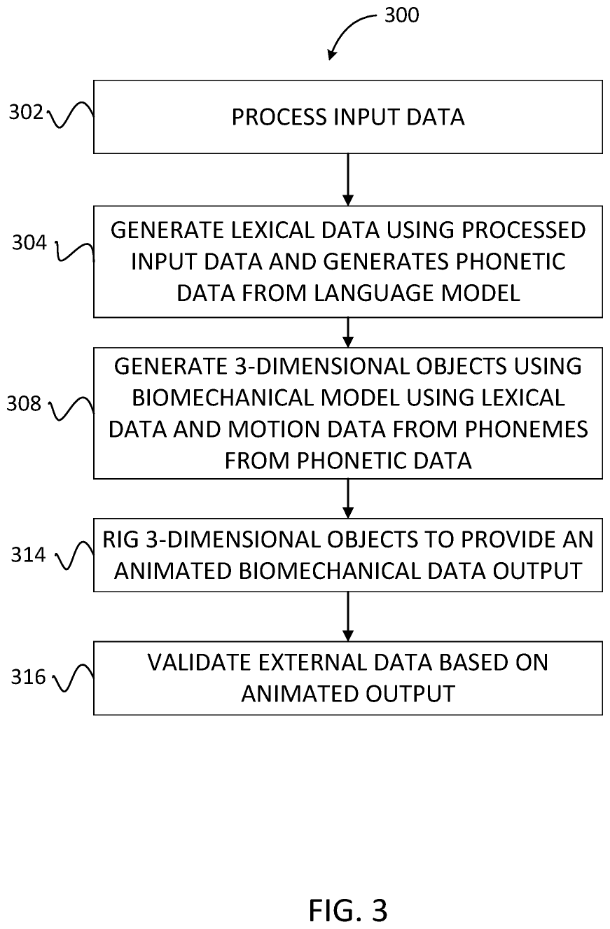

With reference to FIG. 3, FIG. 3 is a flow diagram illustrating an exemplary method 300 for facilitating generation of biomechanical data in accordance with one embodiment of the present disclosure. A processing device such as a user device, a server, a cloud computing service or the like implements the exemplary method 300. The transcriptive biomechanical system can initiate the transcriptive biomechanical method 300 as described herein.

As shown in FIG. 3, in one embodiment, at block 302, a transcriptive biomechanical system obtains input data and verifies whether the input data is in an acoustic data format. At block 302, the biomechanical generating system processes the input data. In one embodiment, the input data is processed to acoustic data and further into language, phonetic, acoustic, and text data. In another embodiment, processing the data can also include processing speech for clarity and interpretability. Denoising includes removing background noise, white noise, unwanted noise, hardware noise, far field noises, or the like, and that step is common in most acoustic processing.

The biomechanical data system, at block 304, uses the acoustic data to generate lexical data. The various types of data can be generated using codex mapping. A suitable codex mapping can be used based on the type of multimodal input. The new data from codex translation can also be generated for use in artificial intelligence methods.

The biomechanical data system, at block 308, maps the language data to a biomechanical model to generate a stack of 3D objects. The 3D objects can be biomechanical objects such as throat, nose, tongue, mouth, cheeks etc. The 3D objects work together to create biological understanding of the lexical data. These 3D objects are mapped in a coordinate system. It should be understood that other data can be used to generate a biomechanical model. For example, the input acoustic data, phonetic data, text data or even language data can be used to generate a biomechanical model.

The biomechanical data system, at block 314, rigs the stack of 3D objects to provide an animated biomechanical data output. The animated biomechanical data output show the stack of 3D objects move the coordinate system to produce the sounds from the acoustic data.

Having described implementations of the present disclosure, an exemplary operating environment in which embodiments of the present technology may be implemented is described below in order to provide a general context for various aspects of the present disclosure. Referring to FIG. 4, an exemplary operating environment for implementing embodiments of the present technology is shown and designated generally as computing device 400. Computing device 400 is but one example of a suitable computing environment and is not intended to suggest any limitation as to the scope of use or functionality of the technology described herein. Neither should the computing device 400 be interpreted as having any dependency or requirement relating to any one or combination of components illustrated.

The technology may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to code that perform particular tasks or implement particular abstract data types. The technology described herein may be practiced in a variety of system configurations, including hand-held devices, consumer electronics, general-purpose computers, more specialty computing devices, etc. The technology described herein may also be practiced in distributed computing environments where tasks are performed by remote-processing devices that are linked through a communications network.

With reference to FIG. 4, computing device 400 includes bus 410 that directly or indirectly couples the following devices: memory 412, one or more processors 414, one or more presentation components 416, input/output (I/O) ports 418, input/output components 420, and illustrative power supply 422. Bus 410 represents what may be one or more busses (such as an address bus, data bus, or combination thereof). Although the various blocks of FIG. 4 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. The inventors recognize that such is the nature of the art, and reiterate that the diagram of FIG. 4 is merely illustrative of an exemplary computing device that can be used in connection with one or more embodiments of the present disclosure. Distinction is not made between such categories as “workstation,” “server,” “laptop,” “hand-held device,” etc., as all are contemplated within the scope of FIG. 4 and reference to “computing device.”

Computing device 400 typically includes a variety of computer-readable media. Computer-readable media can be any available media that can be accessed by computing device 400 and includes both volatile and nonvolatile media, removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 400. Computer storage media does not comprise signals per se. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Memory 412 includes computer storage media in the form of volatile and/or nonvolatile memory. The memory may be removable, non-removable, or a combination thereof. Exemplary hardware devices include solid-state memory, hard drives, optical-disc drives, etc. Computing device 400 includes one or more processors that read data from various entities such as memory 412 or I/O components 420. Presentation component(s) 416 present data indications to a user and/or system or other device. Exemplary presentation components include a display device, speaker, printing component, vibrating component, etc.

I/O ports 418 allow computing device 400 to be logically coupled to other devices including I/O components 420, some of which may be built in. Illustrative components include a microphone, joystick, game pad, satellite dish, scanner, printer, wireless device, etc. The I/O components 420 may provide a natural user and/or system interface (NUI) that processes air gestures, voice, or other physiological inputs generated by a user and/or system. In some instance, inputs may be transmitted to an appropriate network element for further processing. A NUI may implement any combination of speech recognition, touch and stylus recognition, facial recognition, biometric recognition, gesture recognition both on screen and adjacent to the screen, air gestures, head and eye-tracking, and touch recognition associated with displays on the computing device 400. The computing device 400 may be equipped with depth cameras, such as, stereoscopic camera systems, infrared camera systems, RGB camera systems, and combinations of these for gesture detection and recognition. Additionally, the computing device 400 may be equipped with accelerometers or gyroscopes that enable detection of motion.

Aspects of the present technology have been described in relation to particular embodiments, which are intended in all respects to be illustrative rather than restrictive. Alternative embodiments will become apparent to those of ordinary skill in the art to which the present technology pertains without departing from its scope.

Having identified various components utilized herein, it should be understood that any number of components and arrangements may be employed to achieve the desired functionality within the scope of the present disclosure. For example, the components in the embodiments depicted in the figures are shown with lines for the sake of conceptual clarity. Other arrangements of these and other components may also be implemented. For example, although some components are depicted as single components, many of the elements described herein may be implemented as discrete or distributed components or in conjunction with other components, and in any suitable combination and location. Some elements may be omitted altogether. Moreover, various functions described herein as being performed by one or more entities may be carried out by hardware, firmware, and/or software, as described below. For instance, various functions may be carried out by a processor executing instructions stored in memory. As such, other arrangements and elements (e.g., machines, interfaces, functions, orders, and groupings of functions) can be used in addition to or instead of those shown.

Embodiments described herein may be combined with one or more of the specifically described alternatives. In particular, an embodiment that is claimed may contain a reference, in the alternative, to more than one other embodiment. The embodiment that is claimed may specify a further limitation of the subject matter claimed.

The subject matter of embodiments of the technology is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

For purposes of this disclosure, the word “including” has the same broad meaning as the word “comprising,” and the word “accessing” comprises “receiving,” “referencing,” or “retrieving.” Further, the word “communicating” has the same broad meaning as the word “receiving,” or “transmitting” facilitated by software or hardware-based buses, receivers, or transmitters using communication media described herein. In addition, words such as “a” and “an,” unless otherwise indicated to the contrary, include the plural as well as the singular. Thus, for example, the constraint of “a feature” is satisfied where one or more features are present. Also, the term “or” includes the conjunctive, the disjunctive, and both (a or b thus includes either a or b, as well as a and b).

For purposes of a detailed discussion above, embodiments of the present disclosure are described with reference to a distributed computing environment; however, the distributed computing environment depicted herein is merely exemplary. Components can be configured for performing certain embodiments, where the term “configured for” can refer to “programmed to” perform particular tasks or implement particular abstract data types using code. Further, while embodiments of the present disclosure may generally refer to the technical solution environment and the schematics described herein, it is understood that the techniques described may be extended to other implementation contexts.

From the foregoing, it will be seen that this technology is one well adapted to attain all the ends and objects set forth above, together with other advantages which are obvious and inherent to the system and method. It will be understood that certain features and subcombinations are of utility and may be employed without reference to other features and subcombinations. This is contemplated by and is within the scope of the claims.

What is claimed is:

1. A computer-implemented method for generating biomechanical data output, the method comprising:
generating translated data from acoustic, language, or biomechanical inputs and outputs by using codex mapping;
generating a stack of 3-Dimensional objects using a biomechanical model that maps each sound unit or language chunk to 3-Dimensional objects that are “rigged”; and
generating an animated biomechanical data output that moves across coordinate space.

2. The method of claim 1, further comprising:
receiving input data;
validating whether the input data is in an acoustic data format; and
converting the input data into the acoustic or language data format when the input data is not of an acoustic data format.

3. The method of claim 1, further comprising:
performing acoustic processing on the input data, the processing including:
denoising the input data;
segmenting the input data into windows;
identifying a number of speakers from the input data;
classifying the segmented data into chatter and silence; and
processing speech for clarity and interpretability.

4. The method of claim 1, wherein generating the stack of 3-Dimensional objects includes:
obtaining and analyzing a stack of 3-Dimensional biomechanical objects to determine the movement of components in each biomechanical object that produce motion or sound from the lexical, syntactic, semantic data; and
determining coordinates in a 3-Dimensional space for each 3-Dimensional biomechanical object by mapping each of the sound and language units to other units derived by the codex.

5. The method of claim 1, further comprising:
generating a text transcript from the animated biomechanical data output.

6. The method of claim 6, further comprising:
validating a transcript from an external source based on the generated transcripts from the system.

7. The method of claim 1, further comprising:
parsing the input data that goes into the system;
analyzing the at least one of acoustic, phonetic, or language data to deriving natural language data; and
generating the stack of 3-Dimensional objects and analyzing motion of components of the biomechanical objects to determine an underlying medical condition.

8. The method of claim 7, wherein the part of speech is a noun and the underlying medical condition is dementia.

9. The method of claim 1, further comprising:
generating the stack of 3-Dimensional objects using a biomechanical model mapping each sound unit to motion data;

10. The method of claim 1, further comprising:
identifying at least one of a type of speaker from the input data, the type of speaker including gender, age, species of animal, a number of speakers, or other biological sound source; and
generating an animated biomechanical data output for each of the identified sound sources.

11. A computer-implemented method for validating a transcript from an external source, the method comprising:
generating language data from input data using codex mapping, including from multiple sound units or sources;
generating a stack of 3-Dimensional objects using a biomechanical model mapping each sound unit to other codex data, wherein the 3-Dimensional objects include components from the biomechanical model are derived; and
generating an animated biomechanical data output by rigging the stack of 3-Dimensional objects such that the animated biomechanical model of the interaction of the elements produce the language data;
generating a text transcript from the animated biomechanical data output; and
validating a transcript from an external source based on the generated motion from the animated biomechanical data output.

12. The method of claim 11, further comprising:
receiving the input data;
validating whether the input data is of an acoustic data format that is proper; and
converting the input data into the right acoustic data format when the input data is not of a proper acoustic data format.

13. The method of claim 11, further comprising:
identifying a type of speaker from the input data, the type of speaker including gender, age, species of animal or the like.

14. The method of claim 11, further comprising:
identifying a number of speakers from the input data; and
generating an animated biomechanical data output for each of the identified speakers.

15. The method of claim 11, further comprising:
processing the input data for better language understanding; and
modeling data by way of vector modeling.

16. A system comprising:
a memory device; and
a processing device, operatively coupled to the memory device, to perform operations comprising:
generating lexical data from input data using codex mapping, the lexical data including multiple sound units;
parsing the input data into at least one of acoustic phoenetic, or language data;
analyzing the at least one of acoustic, phoenetic, or language data to obtain data matching speech units;
generating a stack of 3-Dimensional objects using a biomechanical model mapping each sound unit to lexical data, wherein the 3-Dimensional objects include components from the biomechanical model; and
analyzing motion of components near lexical data matching the part of the speech to determine an underlying medical condition.

17. The system of claim 16, wherein the part of speech is a noun and the underlying medical condition is dementia.

18. The system of claim 16, further comprising:
generating the stack of 3-Dimensional objects using a biomechanical model mapping each sound unit to motion data;

19. The system of claim 16, further comprising:
identifying at least one of a type of speaker from the input data, the type of speaker including gender, age, species of animal, or a number of speakers; and
generating an animated biomechanical data output for each of the identified speakers

20. The system of claim 16, further comprising:
performing acoustic processing on the input data, the processing including:
denoising the input data;
segmenting the input data into windows;
identifying a number of speakers from the input data;
classifying the segmented data into chatter and silence; and
processing speech for clarity and interpretability.
